(19) United States
(12) Patent Application Publication
(10) Pub. No.: US 2010/0152576 A1
(43) Pub. Date: Jun. 17, 2010

(54) AUXILIARY DEVICE FOR ESTABLISHING A MECHANICAL CONNECTION BETWEEN A MEDICAL IMPLANT AND A TISSUE PART OF A PATIENT

(75) Inventors: Markus Nagel, Nornberg (DE); Ralf Petzold, Erlangen (DE)

Correspondence Address: LERNER GREENBERG STEMER LLP, P O BOX 2480, HOLLYWOOD, FL, 33022-2480, US

(73) Assignee: CAS INNOVATIONS AG, Erlangen (DE)

(21) Appl. No.: 12/377,068

(22) PCT Filed: Aug. 8, 2007
(86) PCT No.: PCT/EP07/07014

§ 371 Date: Feb. 10, 2009

(30) Foreign Application Priority Data
Aug. 10, 2006 (DE) 10 2006 037 602.1

(51) Int. Cl. A61B 5/05; A61B 17/86

(52) U.S. Cl. 600/426; 606/301

(57) ABSTRACT

An auxiliary device for establishing a mechanical connection between a medical implant and a tissue part of a patient, a medical implant for use with such an auxiliary device, and a method for establishing such a mechanical connection. To establish a mechanical connection between a medical implant and a tissue part of a patient in a particularly simple manner, an auxiliary device for establishing a mechanical connection between a medical implant and a tissue part of the patient is provided. A securing element establishes a releasable mechanical connection between the auxiliary device and the tissue part. A marking element is configured to allow its position to be determined with the aid of a contactless locating method. A guide body is connected to the securing element and designed to guide the medical implant to the connection site on the tissue part.

The invention relates to an auxiliary device for establishing a mechanical connection between a medical implant and a tissue part of a patient. The invention further relates to a medical implant for use with such an auxiliary device, and to a method for establishing such a mechanical connection.

In order to stabilize the vertebrae of a human or animal spine, anchoring elements in the form of pedicle screws are known from the prior art. These are inserted into the pedicles of the vertebral arches. Other pedicle screws have, at their free end remote from the threaded end, a device for attachment to a rod, a support or the like.

The insertion of such pedicle screws into the interior of the patient's body and the placement of the pedicle screws in the bone tissue require a particularly high degree of care in order to avoid injuries in the area of the patient's spine.

The prior art includes computer-navigated procedures, especially image-assisted procedures based on a C-arm or computed tomography, by means of which the position of a screwdriver for screwing the pedicle screw into the bone tissue can be monitored inside the body. For this purpose, it is known to arrange an electromagnetic coil on the screwdriver. When this coil is exposed to an electromagnetic field generated for this purpose, a current flow is induced therein. These current signals are detected and, together with other signals serving as references, are used to determine the position of the screwdriver and to navigate it.

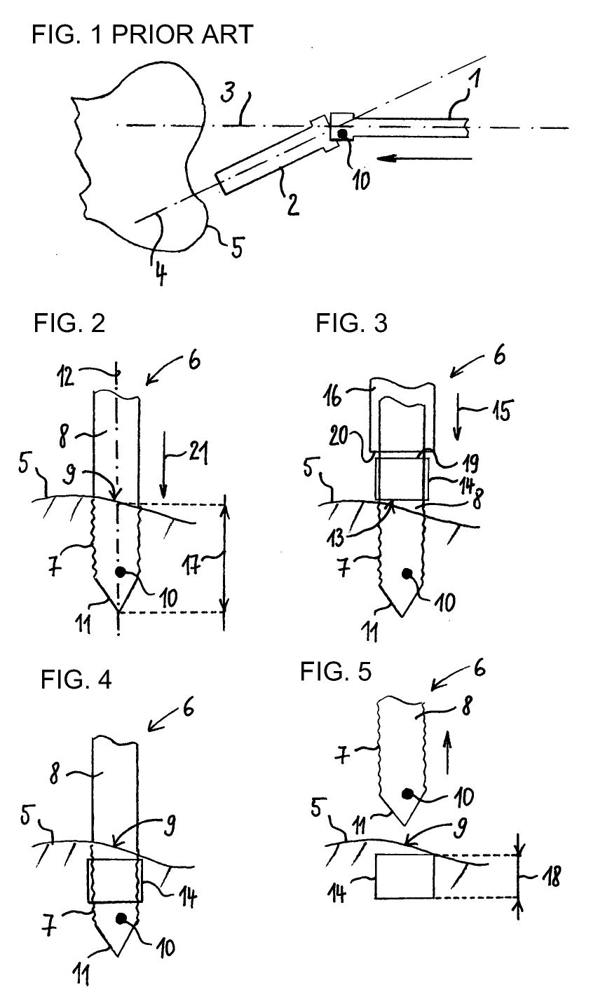

A particular problem is that, as is shown schematically in FIG. 1, although the position of the screwdriver 1 can be set exactly, the position of the pedicle screw 2, and therefore the actual direction 3, may unavoidably deviate from the desired direction 4. If such is the case, precise screwing of the pedicle screw 2 into the bone 5 is not possible. In addition, the wrongly positioned pedicle screw can cause irreparable damage to the bone. During placement of the screw, the operating surgeon is unable to check whether the pedicle screw has in fact been positioned in the planned way, or he is able to do so only by means of an imaging technique which is associated with radiation exposure.

One approach to solving this problem would, for example, be to provide the pedicle screw also with a marking coil. However, this is relatively complicated. It is also undesirable for the marking coils to remain in the patient's body.

It is an object of the present invention to establish a mechanical connection between a medical implant and a tissue part of a patient in a particularly simple manner.

This object is achieved by an auxiliary device for establishing a mechanical connection between a medical implant and a tissue part of a patient, with a securing element for establishing a releasable mechanical connection between the auxiliary device and the tissue part, with a marking element which is designed such that its position can be determined by means of a contactless locating method, and with a guide body which is connected to the securing element and which is designed for guiding the medical implant to the connection site on the tissue part.

This object is also achieved by a medical implant designed for establishing a mechanical connection to a tissue part of a patient, with a guide element which is designed for cooperation with the guide body of an auxiliary device of the kind stated above.

Finally, this object is also achieved by a method for establishing a mechanical connection between a medical implant and a tissue part of a patient, with the following steps: determining the position of the marking element of an auxiliary device, of the kind specified above, by means of a contactless locating method, navigating the auxiliary device to the connection site on the tissue part, establishing a releasable mechanical connection between an auxiliary device and the tissue part, guiding the medical implant to the connection site on the tissue part with the aid of the guide body of the auxiliary device, and establishing the mechanical connection between the medical implant and the tissue part by actuation of the medical implant.

Advantageous embodiments of the invention are set forth in the dependent claims and are explained in more detail below on the basis of an example shown in the drawings, in which:

FIG. 1 shows the placement of a pedicle screw with the aid of a screwdriver (prior art),

FIGS. 2 to 5 show the placement of a pedicle screw according to the invention.

In all the figures, the invention is only shown schematically and with its principal components.

A fundamental idea of the invention is that of using an auxiliary device 6 in order to establish a mechanical connection between a medical implant, specifically a pedicle screw 2, and a tissue part of a patient, specifically a bone tissue 5.

This auxiliary device 6 has a securing element for establishing a releasable mechanical connection between the auxiliary device 6 and the bone 5. The securing element is a cutting thread 7 which is arranged as an outer thread on the main body 8 of the auxiliary device 6. However, instead of a cutting thread, with which a secure connection can be established in a particularly uncomplicated manner, it is also possible to use other securing elements, for example of such a type that a clamped connection is established instead of a screwed connection.

The pin-shaped and cylindrical main body 8 of the auxiliary device 6 at the same time performs the function of the guide body. In other words, it is designed to guide the pedicle screw 2 to the connection site 9 on the bone 5, as is described in detail further below. Moreover, the auxiliary device 6 comprises a marking element, specifically an electromagnetic coil 10, which is designed such that its position can be determined by means of an electromagnetic locating method.

In a first step, the auxiliary device 6 is connected to the bone 5. For this purpose, the end of the main body 8 provided with the cutting thread 7, and provided with a tip 11 with a sharp cutting edge, is pressed into the bone 5. By turning the main body 8 about the axis of rotation 12, which at the same time also represents the longitudinal axis of the main body 8, the cutting thread 7 is then turned into the bone 5 in connection direction 21 (see FIG. 2).

The navigating of the auxiliary device 6 to the bone 5 in the body of the patient, more precisely to the future connection site 9, and the connecting to the bone 5 are performed using the electromagnetic coil 10, which is located entirely within the main body 8 and whose measurement signals are received and processed by a positioning system (not shown) for the purpose of navigation. This therefore permits minimally invasive, computer-navigated placement of the auxiliary device 6 on or in the bone 5 of the patient.

The electromagnetic coil 10 is arranged in the area of the cutting thread 7. This ensures that the coil 10 is located in or on the bone tissue 5 or at least in immediate proximity to the bone tissue 5. This permits particularly exact navigation of the auxiliary device 6 relative to the bone 5, on the basis of the coil signals.

The pedicle screw 2 is then pushed onto the main body 8 of the auxiliary device 6 and is in this way brought quickly and extremely precisely to the connection site 9 on the bone 5 of the patient where the mechanical connection between the pedicle screw 2 and the bone 5 is to be established (see FIG. 3). For this purpose, the pedicle screw 2 is provided with a circular longitudinal opening 19, which cooperates with the main body 8 of the auxiliary device 6. At the connection site 9, the pedicle screw 2 then lies with its connection end 13 on the bone tissue 5 of the patient. At least the connection end 13, but preferably the whole part of the pedicle screw 2 to be secured in the bone 5, also has a cutting thread 14 to permit easier fixing in the bone 5, although, for reasons of clarity, this cutting thread 14 is not shown in detail.

By virtue of the pedicle screw 2 being guided onto and on the main body 8 of the auxiliary device 6, the pedicle screw 2 is automatically located in the desired position, in particular collinear with respect to the puncture channel through the tissue adjacent to the bone 5, along the longitudinal axis 12 of the auxiliary device 6. In other words, the pedicle screw 2 is then arranged in such a way that it only has to be moved in the securing direction 15 towards the bone 5, that is to say in the direction of the longitudinal axis 12. This is done using a suitable actuating tool 16, which is likewise pushed onto the main body 8 and can be moved thereon towards the pedicle screw 2 until contact is made between the actuating tool 16 and that end of the pedicle screw remote from the connection end 13. For this purpose, the actuating tool 16 also has a longitudinal opening 20. When the actuating element 16 and pedicle screw 2 are in engagement with each other, the pedicle screw 2 can be actuated, i.e. screwed into the bone tissue 5, for example by turning the actuating instrument 16 about its longitudinal axis. The longitudinal axis of the actuating element lies on the longitudinal axis 12 of the auxiliary device 6.

The auxiliary device 6 is preferably inserted far enough into the bone tissue 5 to ensure that, when the pedicle screw 2 is screwed in (guided on the main body 8 of the auxiliary device 6), it is possible to avoid the pedicle screw 2 becoming jammed or otherwise deviating from the desired target direction. The depth of screwing 17 of the auxiliary device 6 in the bone tissue 5 preferably corresponds at least to the length 18 of that part of the pedicle screw 2 located within the bone 5 when fitted in place.

After the pedicle screw 2 has been introduced into the bone tissue 5, the actuating instrument 16 can be removed (see FIG. 4). Thereafter, the connection between the auxiliary device 6 and the bone 5 can be released again, and the auxiliary device 6 can be removed from the bone 5 and withdrawn from the patient's body (see FIG. 5). The pedicle screw 2 remains fixed exactly on the bone 5 of the patient.

All of the features set forth in the description, in the appended claims and in the drawing can be considered essential to the invention both individually and also in any desired combination with one another.

### LIST OF REFERENCE SIGNS

1 screwdriver
2 pedicle screw
3 actual direction
4 desired direction
5 bone
6 auxiliary device
7 cutting thread
8 main body
9 connection site
10 coil
11 tip
12 axis of rotation
13 connection end
14 cutting thread
15 securing direction
16 actuating tool
17 depth of screwing
18 length of screwing
19 longitudinal opening
20 longitudinal opening
21 connection direction

1-12. (canceled)

13. An auxiliary device for establishing a mechanical connection between a medical implant and a tissue part of a patient, comprising:
a securing element for establishing a releasable mechanical connection between the auxiliary device and the tissue part at a connection site;
a marking element configured to allow a position thereof to be detected by way of a contactless locating method; and
a guide body connected to said securing element and configured to guide the medical implant to the connection site on the tissue part.

14. The auxiliary device according to claim 13, wherein said securing element is configured for a releasable connection to a bone tissue.

15. The auxiliary device according to claim 13, wherein said securing element is a cutting thread.

16. The auxiliary device according to claims 13, wherein said marking element is disposed in a vicinity of said securing element.

17. The auxiliary device according to claim 13, wherein said marking element is configured for detecting a position thereof by way of an electromagnetic locating method.

18. The auxiliary device according to claim 17, wherein said marking element is an electro-magnetic coil.

19. The auxiliary device according to claim 13, wherein said guide body is configured to enable the medical implant to be pushed onto said guide body and to be moved thereon in a direction towards the connection site.

20. The auxiliary device according to claim 19, wherein said guide body is configured to guide the medical implant in form of a pedicle screw.

21. The auxiliary device according to claim 19, wherein said guide body is configured such that, in addition to the medical implant, a tool for actuating the medical implant can be pushed onto said guide body and moved thereon in the direction towards the connection site.

22. A medical implant, constructed for establishing a mechanical connection to a tissue part of a patient, and comprising a guide element configured for cooperation with the guide body of the auxiliary device according to claim 13.

23. A method for establishing a mechanical connection between a medical implant and a tissue part of a patient, which comprises:
determining a position of a marking element of an auxiliary device according to claim 13 by way of a contactless locating method;
navigating the auxiliary device to the connection site on the tissue part;
establishing a releasable mechanical connection between an auxiliary device and the tissue part;
guiding the medical implant to the connection site on the tissue part with the aid of the guide body of the auxiliary device; and
establishing the mechanical connection between the medical implant and the tissue part by actuation of the medical implant.

24. The method according to claim 23, which further comprises:
releasing the mechanical connection between the auxiliary device and the tissue part; and
removing the auxiliary device.
